(19) United States
(12) Patent Application Publication
(10) Pub. No.: US 2023/0240950 A1
(43) Pub. Date: Aug. 3, 2023

(54) ANHYDROUS COSMETIC COMPOSITIONS, PROCESS OF MANUFACTURING A COSMETIC COMPOSITION AND USE OF THE ANHYDROUS COSMETIC COMPOSITIONS

(71) Applicant: L'OREAL, Paris (FR)

(72) Inventors: Wagner Gomes PEREIRA, Rio de Janeiro (BR); Lais Moreira LIMA, Rio de Janeiro (BR); Angeles FONOLLA MORENO, Clark, NJ (US)

(21) Appl. No.: 18/003,396

(22) PCT Filed: Jun. 30, 2020
(86) PCT No.: PCT/BR2020/050235

§ 371 Date: Dec. 27, 2022

(51) Int. Cl. A61K 8/25; A61K 8/02; A61Q 17/04; A61Q 19/00

(52) U.S. Cl. CPC A61K 8/25; A61K 8/0204; A61Q 17/04; A61Q 19/008; A61K 2800/31; A61K 2800/5922

(57) ABSTRACT

The present invention is related to anhydrous cosmetic compositions having enhanced anti-oiliness properties, dry touch to the skin after the application combined with mate effect, and with a very light, thin and invisible touch on the skin, comprising A) an emollient system, B) a filler system, and C) an UV filter. The present invention is also directed to a process of manufacturing the anhydrous cosmetic compositions and their uses as a sunscreen daily product and for reducing the oiliness of the skin.

### FIELD OF THE INVENTION

The present disclosure is directed to high SPF anhydrous cosmetic compositions. More specifically, the present disclosure is directed to high SPF anhydrous cosmetic compositions in a stick format having enhanced anti-oiliness properties, dry touch to the skin after the application combined with mate effect, and with a very light, thin and invisible touch on the skin, wherein the anhydrous cosmetic compositions comprising A) an emollient system, B) a filler system, and C) an UV filter system. The present invention is also directed to a process of manufacturing the anhydrous cosmetic compositions and their uses as a cosmetic sunscreen daily product and for reducing the oiliness of the skin.

### BACKGROUND OF THE INVENTION

The photoprotection of keratinous materials, including both skin and hair, is considered of great importance in order to protect from sun-damage, sunburn, photo-aging, as well as to decrease the chances of skin cancer development caused by exposure to ultraviolet (“UV”) radiation. There are typically two types of UVA/UVB cosmetic sunscreen compositions used to accomplish photoprotection, namely, inorganic UV filters and organic UV filters.

The degree of UV protection afforded by a cosmetic sunscreen composition is directly related to the amount and type of UV filters contained therein. The higher the amount of UV filters, the greater the degree of UV protection (UVA/UVB). Particularly, cosmetic sunscreen compositions must provide good protection against the sun, a measure of which is the Sun Protection Factor (SPF) value, yet have satisfactory sensory perception, such as a smooth but not greasy feel upon application.

Usually, cosmetic sunscreen products may be in the form of lotions, milks, creams, gels, gel creams, foams, sprays and sticks. Such products can be anhydrous or in the form of emulsions, generally containing cosmetic sunscreen actives that are solubilized, emulsified, or dispersed in a vehicle, which is topically applied onto the skin. The cosmetic sunscreen actives, typically through the aid of polymers and other ingredients included in the vehicle, form a thin, protective, and often water-resistant layer on the skin.

Hydrophobic silica is commonly known in the cosmetic industry for providing anti-oiliness and anti-acne effects, including in cosmetic sunscreen compositions. However, the maximum concentration of silica in a composition is limited, due to its high hydrophobic properties, thereby rendering the composition instable when high concentrations of silica are applied. In this sense, cosmetic sunscreen compositions are usually limited to have about 0.5% by weight of silica, relative to the total weight of the composition.

Thus, there has been a need for cosmetic sunscreen compositions having higher concentrations of silica, to enhance the anti-oiliness, dry touch and matte effect thereof. The challenge of incorporating high concentrations of silica in the cosmetic sunscreen composition is not only limited due to the hydrophobic nature of the silica, but there is also the challenge on formulating stable compositions while preserving satisfactory properties of the product, such as hardness, high SPF and dry touch to the skin after application combined with matt effect.

Considering the current drawbacks of the state of the art and the difficulties to overcome them, the inventors formulated an anhydrous cosmetic composition that enables the high concentration of hydrophobic silica by combining an emollient system, a filler system and an UV filter system. The anhydrous cosmetic compositions of the present invention having high concentration of silica surprisingly showed enhanced anti-oiliness effect, high SPF values, dry touch to the skin after application combined with matte effect, and with a very light, thin and invisible touch on the skin.

### SUMMARY OF THE INVENTION

The present invention is directed to anhydrous cosmetic compositions having enhanced anti-oiliness effect, high SPF values, dry touch to the skin after application combined with matte effect, and with a very light, thin and invisible touch on the skin, comprising an emollient system, a filler system and an UV filter system. The present disclosure is also directed to a process of manufacturing the anhydrous cosmetic compositions and uses of the anhydrous cosmetic compositions.

Particularly, the present invention relates to the use of the cosmetic composition for the manufacture of a product to be used as a sunscreen daily product and for reducing the oiliness of the skin.

Other features and advantages of the present invention will be apparent from the following more detailed description of the desirable embodiments which illustrates, by way of example, the principles of the invention.

### DETAILED DESCRIPTION OF THE INVENTION

The anhydrous cosmetic compositions of the present invention can comprise, consist of, or consist essentially of the essential elements and limitations of the present invention described herein, as well as any of the additional or optional ingredients, components, or limitations described herein.

The terms “a”, “an”, and “the” are understood to encompass the plural as well as the singular.

As used herein, the expression “at least one” means one or more and thus includes individual components as well as mixtures/combinations.

All ranges and values disclosed herein are inclusive and combinable. For examples, any value or point described herein that falls within a range described herein can serve as a minimum or maximum value to derive a sub-range, etc.

The term “anhydrous” as used herein means that the cosmetic composition of the present invention, and the essential or optional components thereof, are substantially free of added or free water. From a formulation standpoint, this means that the cosmetic composition of the present invention contains less than about 5%, preferably less than about 3%, more preferably less than about 2% by weight, even more preferably less than about 1% by weight of free or added water.

In an embodiment, the anhydrous cosmetic composition of the present invention comprises:

(a) at least 1% by weight of an emollient system, relative to the total weight of the composition;

(b) at least 0.5% by weight of a filler system, relative to the total weight of the composition; and

(c) an UV filter system.

The composition according to the invention presents enhanced anti-oiliness properties, high SPF values, dry touch to the skin after application combined with matte effect, and, surprisingly, with a very light, thin and invisible touch on the skin, given the strong synergy between the emollient system and the filler system.

In a preferred embodiment, the emollient system in the anhydrous cosmetic composition of the present invention is from about 1% by weight to about 35% by weight, relative to the total weight of the composition, more preferably from about 5% by weight to about 30% by weight, even more preferably from about 10% by weight to about 25% by weight, most preferably from about 15% by weight to about 25% by weight, relative to the total weight of the composition.

The emollient system of the present invention comprises at least one emollient selected from the group consisting of diisopropyl adipate, isononyl isononanoate, dicaprylyl ether, caprylic/capric triglyceride, dicaprylyl carbonate, isopropyl lauroyl sarcosinate, dibutyl adipate, isoamyl caprate-caprylate, and mixtures thereof.

In a preferred embodiment of the invention, the emollient system comprises a mixture of the emollients isononyl isononanoate, dicaprylyl ether and diisopropyl adipate, which mixture has a weight ratio of 10:10:1.

In a preferred embodiment, the amount of the filler system in the anhydrous cosmetic composition of the present invention is from about 0.5% by weight to about 20% by weight, relative to the total weight of the composition, more preferably from about 5% by weight to about 15% by weight, even more preferably from about 10% by weight to about 15% by weight, relative to the total weight of the composition.

The filler system of the present invention comprising at least one filler selected from the group consisting of silica, silica silylate, talc, mica, cellulose and mixtures thereof. As regards cellulose, this filler can be used in synergy or as a substitute for talc or mica.

In a preferred embodiment of the invention, the filler system comprises a mixture of the fillers silica, silica silylate, talc, and mica, which mixture has a weight ratio of 2:1:1.5:1.5.

In a preferred embodiment, the amount of the UV filter system in the anhydrous cosmetic composition of the present invention is from about 0.1% by weight to about 40% by weight, relative to the total weight of the composition, more preferably from about 5% by weight to about 35% by weight, even more preferably from about 10% by weight to about 30% by weight, most preferably from about 15% by weight to about 25% by weight, relative to the total weight of the composition.

The UV filter system of the present invention may comprise at least one UV filter selected from the group of inorganic UV filters and organic UV filters, and mixtures thereof. Also, any other organic UV filter that is oil soluble or inorganic UV filter can be used in this composition.

In a preferred embodiment, the UV filter system comprise butyl methoxydibenzoylmethane, ethylhexyl salicylate, ethylhexyl triazone, octocrylene, drometrizole trisiloxane, bis-ethylhexyloxyphenol methoxyphenyl triazine, diethylamino hydroxybenzoyl hexyl benzoate, drometrizole trisiloxane or mixtures thereof.

In a preferred embodiment, the UV filter system of the present invention comprises a mixture of butyl methoxydibenzoylmethane, ethylhexyl salicylate, ethylhexyl triazone, octocrylene, drometrizole trisiloxane, and bis-ethylhexyloxyphenol methoxyphenyl triazine, which mixture has a weight ratio of 2:2.5:1.75:1:1.

In a preferred embodiment, the anhydrous cosmetic composition of the present invention comprises additional fatty compounds selected from the group of oils, waxes, fatty acids, fatty alcohols, and mixtures thereof. Said additional fatty compounds are preferably in an amount of at least about 20% by weight, relative to the total weight of the composition, preferably ranging from 20% by weight to 70% by weight, relative to the total weight of composition, more preferably from about 25% by weight to about 65% by weight, even more preferably from about 30% by weight to about 60% by weight, relative to the total weight of the composition. In a preferred embodiment, the additional fatty compounds are selected from the group consisting of synthetic esters, waxes, and mixtures thereof, preferably being selected from the group consisting of ethylhexyl palmitate, diisostearyl malate, polyethylene, or mixtures thereof.

In another preferred embodiment, the composition of the present invention presents a SPF of at least 30, preferably 30, 50, 60, 70, 80, 90 or 100.

The anhydrous cosmetic composition of the invention can be used as a product for the skin and may be in the form of lotions, milks, creams, gels, gel creams, foams, sprays and sticks.

In a preferred embodiment, the anhydrous cosmetic composition of the present invention is in the form of a stick.

In another preferred embodiment, the present invention is related to the use of a composition for manufacturing a product for preventing sunburn, which can be used as cosmetic daily product, preferably in the form of a stick.

The anhydrous cosmetic composition of the present invention can also be used for the manufacture of a cosmetic product for reducing the oiliness of the skin, preferably human skin, more preferably human face skin.

The present invention is also related to a process of manufacturing an anhydrous cosmetic composition that provides for the consumer the properties described above, wherein the anhydrous cosmetic composition comprises:

(a) at least 1% by weight of an emollient system, relative to the total weight of the composition;

(b) at least 0.5% by weight of a filler system, relative to the total weight of the composition; and

(c) an UV filter system.

The process according to the present invention comprises the steps of:

(a) mixing the emollient system with the UV filter system;

(b) optionally, incorporating additional ingredients to the mixture of step (a);

(c) incorporating the filler system to the mixture of step (b); and

(d) homogenizing the mixture of step (c).

### Emollient System

The emollient system according to the present invention comprises dry emollients, preferably, at least one emollient selected from the group consisting of diisopropyl adipate, isononyl isononanoate, dicaprylyl ether, caprylic/capric triglyceride, dicaprylyl carbonate, isopropyl lauroyl sarcosinate, dibutyl adipate, isoamyl caprate-caprylate and mixtures thereof, wherein the emollient system in the anhydrous cosmetic composition is from about 1% by weight to about 35% by weight, relative to the total weight of the composition.

In a preferred embodiment, the emollient system of the anhydrous cosmetic composition is a mixture of diisopropyl adipate, isononyl isononanoate and dicaprylyl ether.

### Filler System

The filler system according to the present invention comprises at least one filler selected from the group consisting of silica, silica silylate, talc, mica, cellulose, zea may (corn) starch, magnesium oxide, nylon-12, nylon-66, cellulose, talc (and) methicone, talc (and) dimethicone, perlite, sodium silicate, pumice, PTFE, polymethyl methacrylate, oryza sativa (rice) starch, aluminum starch octenylsuccinate, potato starch modified, alumina, calcium sodium borosilicate, magnesium carbonate, dimethicone/vinyl dimethicone crosspolymer, sodium carboxylmethyl starch and mixtures thereof.

Particulaly, the “silica silylate” according to the present invention is a porous material obtained by replacing (by drying) the liquid component of a silica gel with air. Silica aerogels are generally synthesized via a sol-gel process in a liquid medium and then dried, usually by extraction with a supercritical fluid, such as, but not limited to, supercritical carbon dioxide (CO₂). This type of drying makes it possible to avoid shrinkage of the pores and of the material. The sol-gel process and the various drying processes are described in detail in Brinker, C. J., and Scherer, G. W., Sol-Gel Science: New York: Academic Press, 1990. The silica silylate according to the present invention, is a hydrophobic silica aerogel.

In a preferred embodiment, the filler system of the anhydrous cosmetic composition is a mixture of silica, silica silylate, talc, and mica, wherein the filler system in the anhydrous cosmetic composition is from 0.5% by weight to 20% by weight, relative to the total weight of the composition.

### UV Filter System

The composition according to the present invention comprises a UV filter system. The UV filter system may comprise at least one UV filter selected from the group of inorganic UV filters, organic UV filters and mixtures thereof.

The composition, according to the present invention, may comprise the UV filter system in an amount of from about 0.1% by weight to about 40% by weight, preferably from about 1% by weight to about 35% by weight, more preferably from about 1% by weight to about 30% by weight, even more preferably from about 1% by weight to about 25% by weight, in relation to the total weight of the composition.

### Inorganic UV Filters

The composition, according to the present invention, comprise a UV filter system comprising at least one inorganic UV filter. If two or more inorganic UV filters are used, they may be the same or different.

The inorganic UV filter used for the present invention may be active in the UV-A and/or UV-B region. The inorganic UV filter may be hydrophilic and/or lipophilic. The inorganic UV filter is in some embodiments insoluble in solvents, such as water, and ethanol commonly used in cosmetics.

It is in some embodiments desirable that the inorganic UV filter be in the form of a fine particle such that the mean (primary) particle diameter thereof ranges from about 1 nm to about 50 nm, and in some embodiments from about 5 nm to about 40 nm, and in some embodiments from about 10 nm to about 30 nm. The mean (primary) particle size or mean (primary) particle diameter here is an arithmetic mean diameter.

The inorganic UV filter can be selected from the group consisting of silicon carbide, metal oxides which may or may not be coated, and mixtures thereof. And in some embodiments, the inorganic UV filters are selected from pigments (mean size of the primary particles: generally from about 5 nm to about 50 nm, and in some embodiments from about 10 nm to about 50 nm) formed of metal oxides, such as, for example, pigments formed of titanium oxide (amorphous or crystalline in the rutile and/or anatase form), iron oxide, zinc oxide, zirconium oxide, or cerium oxide, which are all UV photoprotective agents that are well known per se. And in some embodiments, the inorganic UV filters are selected from titanium oxide, zinc oxide, and, in some embodiments, titanium oxide.

The inorganic UV filter may or may not be coated. The inorganic UV filter may have at least one coating. The coating may comprise at least one compound selected from the group consisting of alumina, silica, aluminum hydroxide, silicones, silanes, fatty acids or salts thereof (such as sodium, potassium, zinc, iron, or aluminum salts), fatty alcohols, lecithin, amino acids, polysaccharides, proteins, alkanolamines, waxes, such as beeswax, (meth)acrylic polymers, organic UV filters, and (per)fluoro compounds. It is in some embodiments desirable for the coating to include at least one organic UV filter. As the organic UV filter in the coating, a dibenzoylmethane derivative, such as butyl methoxydibenzoylmethane (Avobenzone) and 2,2′-Methylenebis[6-(2H-Benzotriazol-2-yl)-4-(I,I,3,3-Tetramethyl-Butyl) Phenol] (Methylene Bis-Benzotriazolyl Tetramethylbutylphenol), such as marketed as “TINOSORB M” by BASF, may be desirable.

In a known manner, the silicones in the coating(s) may be organosilicon polymers or oligomers comprising a linear or cyclic and branched or cross-linked structure, of variable molecular weight, obtained by polymerization and/or polycondensation of suitable functional silanes and essentially composed of repeated main units in which the silicon atoms are connected to one another via oxygen atoms (siloxane bond), optionally substituted hydrocarbon radicals being connected directly to said silicon atoms via a carbon atom.

The term “silicones” also encompasses silanes necessary for their preparation, in particular alkylsilanes.

The silicones used for the coating(s) can be and in some embodiments are selected from the group consisting of alkylsilanes, polydialkylsiloxanes, and polyalkylhydrosiloxanes. And in some embodiments still, the silicones are selected from the group consisting of octyltrimethylsilane, polydimethylsiloxanes, and polymethylhydrosiloxanes.

Of course, the inorganic UV filters made of metal oxides may, before their treatment with silicones, have been treated with other surfacing agents, in particular with cerium oxide, alumina, silica, aluminum compounds, silicon compounds, or their mixtures. The coated inorganic UV filter may have been prepared by subjecting the inorganic UV filter to one or more surface treatments of a chemical, electronic, mechano-chemical, and/or mechanical nature with any of the compounds as described above, as well as polyethylene waxes, metal alkoxides (titanium or aluminum alkoxides), metal oxides, sodium hexametaphosphate, and those shown, for example, in Cosmetics & Toiletries, February 1990, Vol. 105, pp. 53-64.

The coated inorganic UV filters may be titanium oxides coated: with silica, such as the product “Sun veil” from Ikeda, and “Sunsil TIN 50” from Sunjin Chemical; with silica and with iron oxide, such as the product “Sunveil F” from Ikeda; with silica and with alumina, such as the products “Microtitanium Dioxide MT 500 SA” from Tayca, “Tioveil” from Tioxide, and “Mirasun TiW 60” from Rhodia; with alumina, such as the products “Tipaque TTO-55 (B)” and “Tipaque TTO-55 (A)” from Ishihara, and “UVT 14/4” from Kemira; with alumina and with aluminum stearate, such as the product “Microtitanium Dioxide MT 100 T, MT 100 TX, MT 100 Z or MT-01” from Tayca, the products “Solaveil CT-10 W” and “Solaveil CT 100” from Uniqema, and the product “Eusolex T-AVO” from Merck; with alumina and with aluminum laurate, such as the product “Microtitanium Dioxide MT 100 S” from Tayca; with iron oxide and with iron stearate, such as the product “Microtitanium Dioxide MT 100 F” from Tayca; with zinc oxide and with zinc stearate, such as the product “BR351” from Tayca; with silica and with alumina and treated with a silicone, such as the products “Microtitanium Dioxide MT 600 SAS”, “Microtitanium Dioxide MT 500 SAS”, and “Microtitanium Dioxide MT 100 SAS” from Tayca; with silica, with alumina and with aluminum stearate and treated with a silicone, such as the product “STT-30-DS” from Titan Kogyo; with silica and treated with a silicone, such as the product “UV-Titan X 195” from Kemira; with alumina and treated with a silicone, such as the products “Tipaque TTO-55 (S)” from Ishihara or “UV Titan M 262” from Kemira; with triethanolamine, such as the product “STT-65-S” from Titan Kogyo; with stearic acid, such as the product “Tipaque TTO-55 (C)” from Ishihara; or with sodium hexametaphosphate, such as the product “Microtitanium Dioxide MT 150 W” from Tayca. Other titanium oxide pigments treated with a silicone are, and in some embodiments TiO₂ treated with octyltrimethylsilane and for which the mean size of the individual particles is from 25 and 40 nm, such as that marketed under the trademark “T 805” by Degussa Silices, TiO2 treated with a polydimethylsiloxane and for which the mean size of the individual particles is 21 nm, such as that marketed under the trademark “70250 Cardre UF TiO2Si3” by Cardre, and anatase/rutile TiO2 treated with a polydimethylhydrosiloxane and for which the mean size of the individual particles is 25 nm, such as that marketed under the trademark “Microtitanium Dioxide USP Grade Hydrophobic” by Color Techniques.

And in some embodiments, the following coated TiO2 can be used as the coated inorganic UV filter: Stearic acid (and) Aluminum Hydroxide (and) TiO2, such as the product “MT-100 TV” from Tayca, with a mean primary particle diameter of 15 nm; Dimethicone (and) Stearic Acid (and) Aluminum Hydroxide (and) TiO2, such as the product “S A-TTO-S4” from Miyoshi Kasei, with a mean primary particle diameter of 15 nm; Silica (and) TiO2, such as the product “MT-100 WP” from Tayca, with a mean primary particle diameter of 15 nm; Dimethicone (and) Silica (and) Aluminum Hydroxide (and) TiO2, such as the product “MT-Y02” and “MT-Y-110 M3S” from Tayca, with a mean primary particle diameter of 10 nm; Dimethicone (and) Aluminum Hydroxide (and) TiO2, such as the product “SA-TTO-S3” from Miyoshi Kasei, with a mean primary particle diameter of 15 nm; Dimethicone (and) Alumina (and) TiO2, such as the product “UV TITAN MI 70” from Sachtleben, with a mean primary particle diameter of 15 nm;. and Silica (and) Aluminum Hydroxide (and) Alginic Acid (and) TiO2, such as the product “MT- 100 AQ” from Tayca, with a mean primary particle diameter of 15 nm. In terms of UV filtering ability, TiO2 coated with at least one organic UV filter is more desirable. For example, Avobenzone (and) Stearic Acid (and) Aluminum Hydroxide (and) TiO2, such as the product “HXMT-100ZA” from Tayca, with a mean primary particle diameter of 15 nm, can be used.

The uncoated titanium oxide pigments are, for example, marketed by Tayca under the trademarks “Microtitanium Dioxide MT500B” or “Microtitanium Dioxide MT600B”, by Degussa under the trademark “P 25”, by Wacker under the trademark “Oxyde de titane transparent PW”, by Miyoshi Kasei under the trademark “UFTR”, by Tomen under the trademark “ITS” and by Tioxide under the trademark “Tioveil AQ”. The uncoated zinc oxide pigments are, for example, those marketed under the trademark “Z-cote” by Sunsmart; those marketed under the trademark “Nanox” by Elementis; and those marketed under the trademark “Nanogard WCD 2025” by Nanophase Technologies. The coated zinc oxide pigments are, for example, those marketed under the trademark “Oxide Zinc CS-5” by Toshiba (ZnO coated with polymethylhydrosiloxane); those marketed under the trademark “Nanogard Zinc Oxide FN” by Nanophase Technologies (as a 40% dispersion in Finsolv TN, C12-C15 alkyl benzoate); those marketed under the trademark “Daitopersion Zn-30” and “Daitopersion Zn-50” by Daito (dispersions in oxyethylenated polydimethylsiloxane/cyclopolymethylsiloxane comprising 30% or 50% of zinc nano-oxides coated with silica and polymethylhydrosiloxane); those marketed under the trademark “NFD Ultrafine ZnO” by Daikin (ZnO coated with phosphate of perfiuoroalkyl and a copolymer based on perfluoroalkylethyl as a dispersion in cyclopentasiloxane); those marketed under the trademark “SPD-Z1” by Shin-Etsu (ZnO coated with a silicone-grafted acrylic polymer dispersed in cyclodimethylsiloxane); those marketed under the trademark “Escalol Z100” by ISP (alumina-treated ZnO dispersed in an ethylhexyl methoxycinnamate/PVP-hexadecene copolymer/methicone mixture); those marketed under the trademark “Fuji ZnO-SMS-10” by Fuji Pigment (ZnO coated with silica and polymethylsilsesquioxane); and those marketed under the trademark “Nanox Gel TN” by Elementis (ZnO dispersed at 55% in C₁₂-C₁₅ alkyl benzoate with hydroxystearic acid polycondensate). The uncoated cerium oxide pigments are marketed, for example, under the trademark “Colloidal Cerium Oxide” by Rhone-Poulenc.

The uncoated iron oxide pigments are, for example, marketed by Arnaud under the trademarks “Nanogard WCD 2002 (FE 45B)”, “Nanogard Iron FE 45 BL AQ”, “Nanogard FE 45R AQ”, and “Nanogard WCD 2006 (FE 45R)”, or by Mitsubishi under the trademark “TY-220”.

The coated iron oxide pigments are, for example, marketed by Arnaud under the trademarks “Nanogard WCD 2008 (FE 45B FN)”, “Nanogard WCD 2009 (FE 45B 556)”, “Nanogard FE 45 BL 345”, and “Nanogard FE 45 BL”, or by BASF under the trademark “Oxyde de fer transparent”.

Mention may also be made of mixtures of metal oxides, in particular, of titanium dioxide and of cerium dioxide, including a mixture of equal weights of titanium dioxide coated with silica and of cerium dioxide coated with silica, such as marketed by Ikeda under the trademark “Sunveil A”, and also a mixture of titanium dioxide and of zinc dioxide coated with alumina, with silica and with silicone, such as the product “M 261” marketed by Kemira, or coated with alumina, with silica and with glycerol, such as the product “M 211” marketed by Kemira.

Coated inorganic UV filters are desirable, because the UV filtering effects of the inorganic UV filters can be enhanced. In addition, the coating(s) may help uniformly or homogeneously disperse the UV filters in the composition, according to the present invention.

### Organic UV Filters

The composition, according to the present invention, comprises a UV filter system comprising at least one organic UV filter. If two or more organic UV filters are used, they may be the same or different.

The organic UV filter used for the present invention may be active in the UV-A and/or UV-B region. The organic UV filter may be hydrophilic and/or lipophilic.

The organic UV filter may be solid or liquid. The terms “solid” and “liquid” mean solid and liquid, respectively, at 25° C. under 1 atm.

The organic UV filter can be selected from the group consisting of anthranilic compounds; dibenzoylmethane compounds; cinnamic compounds; salicylic compounds; camphor compounds; benzophenone compounds; β,β-diphenylacrylate compounds; triazine compounds; benzotriazole compounds; benzalmalonate compounds; benzimidazole compounds; imidazoline compounds; bis-benzoazolyl compounds; p-aminobenzoic acid (PABA) compounds; methylenebis(hydroxyphenylbenzotriazole) compounds; benzoxazole compounds; screening polymers and screening silicones; dimers derived from a-alkylstyrene; 4,4-diarylbutadienes compounds; guaiazulene and derivatives thereof; rutin and derivatives thereof; flavonoids; bioflavonoids; oryzanol and derivatives thereof; quinic acid and derivatives thereof; phenols; retinol; cysteine; aromatic amino acids; peptides having an aromatic amino acid residue; and mixtures thereof.

Mention may be made, as examples of the organic UV filter(s), of those denoted below under their INCI names, and mixtures thereof. Anthranilic compounds: menthyl anthranilates, such as marketed under the trademark “Neo Heliopan MA” by Haarmann and Reimer. The dibenzoylmethane compounds: Butyl methoxydibenzoylmethane, such as marketed in particular under the trademark “Parsol 1789” by Hoffmann-La Roche; and isopropyl dibenzoylmethane. Cinnamic compounds: Ethylhexyl methoxycinnamate, such as marketed in particular under the trademark “Parsol MCX” by Hoffmann-La Roche; isopropyl methoxycinnamate; isopropoxy methoxycinnamate; isoamyl methoxycinnamate, such as marketed under the trademark “Neo Heliopan E 1000” by Haarmann and Reimer; cinoxate (2-ethoxyethyl-4-methoxy cinnamate); DEA methoxycinnamate; diisopropyl methylcinnamate; and glyceryl ethylhexanoate dimethoxycinnamate. Salicylic compounds: Homosalate (homomentyl salicylate), such as marketed under the trademark “Eusolex HMS” by Rona/EM Industries; ethylhexyl salicylate, such as marketed under the trademark “Neo Heliopan OS” by Haarmann and Reimer; glycol salicylate; butyloctyl salicylate; phenyl salicylate; dipropyleneglycol salicylate, such as marketed under the trademark “Dipsal” by Scher; and TEA salicylate, such as marketed under the trademark “Neo Heliopan TS” by Haarmann and Reimer. Camphor compounds, in particular, benzylidenecamphor derivatives: 3-benzylidene camphor, such as manufactured under the trademark “Mexoryl SD” by Chimex; 4-methylbenzylidene camphor, such as marketed under the trademark “Eusolex 6300” by Merck; benzylidene camphor sulfonic acid, such as manufactured under the trademark “Mexoryl SL” by Chimex; camphor benzalkonium methosulfate, such as manufactured under the trademark “Mexoryl SO” by Chimex; terephthalylidene dicamphor sulfonic acid, such as manufactured under the trademark “Mexoryl SX” by Chimex; and polyacrylamidomethyl benzylidene camphor, such as manufactured under the trademark “Mexoryl SW” by Chimex. Benzophenone compounds: Benzophenone-1 (2,4-dihydroxybenzophenone), such as marketed under the trademark “Uvinul 400” by BASF; benzophenone-2 (Tetrahydroxybenzophenone), such as marketed under the trademark “Uvinul D50” by BASF; Benzophenone-3 (2-hydroxy-4-methoxybenzophenone) or oxybenzone, such as marketed under the trademark “Uvinul M40” by BASF; benzophenone-4 (hydroxymethoxy benzophonene sulfonic acid), such as marketed under the trademark “Uvinul MS40” by BASF; benzophenone-5 (Sodium hydroxymethoxy benzophenone Sulfonate); benzophenone-6 (dihydroxy dimethoxy benzophenone); such as marketed under the trademark “Helisorb 11” by Norquay; benzophenone-8, such as marketed under the trademark “Spectra-Sorb UV-24” by American Cyanamid; benzophenone-9 (Disodium dihydroxy dimethoxy benzophenonedisulfonate), such as marketed under the trademark “Uvinul DS-49” by BASF; and benzophenone-12, and n-hexyl 2-(4-diethylamino-2-hydroxybenzoyl)benzoate (such as UVINUL A+ by BASF). β,β-Diphenylacrylate compounds: Octocrylene, such as marketed in particular under the trademark “Uvinul N539” by BASF; and Etocrylene, such as marketed in particular under the trademark “Uvinul N35” by BASF. Triazine compounds: Diethylhexyl butamido triazone, such as marketed under the trademark “Uvasorb HEB” by Sigma 3V; 2,4,6-tris(dineopentyl 4′-aminobenzalmalonate)-s-triazine, bis-ethylhexyloxyphenol methoxyphenyl triazine, such as marketed under the trademark «TINOSORB S» by CIBA GEIGY, and ethylhexyl triazone, such as marketed under the trademark «UVTNUL T150» by BASF. Benzotriazole compounds, in particular, phenylbenzotriazole derivatives: 2-(2H-benzotriazole-2-yl)-6-dodecyl-4-methylpheno, branched and linear; and those described in USP 5240975. Benzalmalonate compounds: Dineopentyl 4′-methoxybenzalmalonate, and polyorganosiloxane comprising benzalmalonate functional groups, such as polysilicone-15, such as marketed under the trademark “Parsol SLX” by Hoffmann-LaRoche. Benzimidazole compounds, in particular, phenylbenzimidazole derivatives: Phenylbenzimidazole sulfonic acid, such as marketed in particular under the trademark “Eusolex 232” by Merck, and disodium phenyl dibenzimidazole tetrasulfonate, such as marketed under the trademark “Neo Heliopan AP” by Haarmann and Reimer. Imidazoline compounds: Ethylhexyl dimethoxybenzylidene dioxoimidazoline propionate. Bis-benzoazolyl compounds: The derivatives as described in EP-669,323 and U.S. Pat. No. 2,463,264. Para-aminobenzoic acid compounds: PABA (p-aminobenzoic acid), ethyl PABA, Ethyl dihydroxypropyl PABA, pentyl dimethyl PABA, ethylhexyl dimethyl PABA, such as marketed in particular under the trademark “Escalol 507” by ISP, glyceryl PABA, and PEG-25 PABA, such as marketed under the trademark “Uvinul P25” by BASF. Methylene bis-(hydroxyphenylbenzotriazol) compounds, such as 2,2′-methylenebis[6-(2H-benzotriazol-2-yl)-4-methyl-phenol], such as marketed in the solid form under the trademark “Mixxim BB/200” by Fairmount Chemical, 2,2′-methylenebis[6-(2H-benzotriazol-2-yl)-4-(I,I,3,3-tetramethylbutyl)phenol], such as marketed in the micronized form in aqueous dispersion under the trademark “Tinosorb M” by BASF, or under the trademark “Mixxim BB/100” by Fairmount Chemical, and the derivatives as described in U.S. Pat. Nos. 5,237,071 and 5,166,355, GB-2,303,549, DE-197,26,184, and EP-893,119, and Drometrizole trisiloxane, such as marketed under the trademark “Silatrizole” by Rhodia Chimie or—“Mexoryl XL” by L'Oréal. Benzoxazole compounds: 2,4-bis[5-I(dimethylpropyl)benzoxazol-2-yl-(4-phenyl)imino]-6-(2-ethylhexyl)imino-I,3,5-triazine, such as marketed under the trademark of Uvasorb K2A by Sigma 3V.

Screening polymers and screening silicones: The silicones described in WO 93/04665. Dimers derived from a-alkylstyrene: The dimers described in DE-19855649. 4,4-Diarylbutadiene compounds: I,I,-dicarboxy(2,2′-dimethylpropyl)-4,4-diphenylbutadiene.

It is in some embodiments desirable that the organic UV filter(s) be selected from the group consisting of: butyl methoxydibenzoylmethane, ethylhexyl methoxycinnamate, homosalate, ethylhexyl salicylate, octocrylene, phenylbenzimidazole sulfonic acid, benzophenone-3, benzophenone-4, benzophenone-5, n-hexyl 2-(4-diethylamino-2-hydroxybenzoyl)benzoate, I,r-(I,4-piperazinediyl)bis[I-[2-[4-(diethylamino)-2-hydroxybenzoyl]phenyl]-methanone 4-methylbenzylidene camphor, terephthalylidene dicamphor sulfonic acid, disodium phenyl dibenzimidazole tetrasulfonate, ethylhexyl triazone, bis-ethylhexyloxyphenol methoxyphenyl triazine, diethylhexyl butamido triazone, 2,4,6-tris(dineopentyl 4′-aminobenzalmalonate)- s-triazine, 2,4,6-tris(diisobutyl 4′-aminobenzalmalonate)-s-triazine, 2,4-bis-(n-butyl 4′-aminobenzalmalonate)-6-[(3-{1,3,3,3-tetramethyl-1-[(trimethylsilyloxy]-disiloxanyl}propyl)amino]-s-triazine, 2,4,6-tris-(di-phenyl)-triazine, 2,4,6-tris-(ter-phenyl)-triazine, methylene bis-benzotriazolyl tetramethylbutylphenol, drometrizole trisiloxane, polysilicone-15, dineopentyl 4′-methoxybenzalmalonate, I,I-dicarboxy(2,2′-dimethylpropyl)-4,4-diphenylbutadiene, 2,4-bis[5-I (dimethylpropyl)benzoxazol-2-yl-(4-phenyl)imino]-6-(2-ethylhexyl)imino-I,3,5-triazine, camphor benzylkonium methosulfate, and mixtures thereof.

### Additional Fatty Compounds

In addition to the constituents of the anhydrous cosmetic composition described above, the composition of the present invention can also comprise additional fatty compounds selected from oils, waxes, fatty acids, fatty alcohols, and mixtures thereof.

The proportion of additional fatty compounds in the anhydrous cosmetic composition according to the invention is generally of at least about 20% by weight, relative to the total weight of the composition, preferably ranging from 20% by weight to 70% by weight, more preferably from about 25% by weight to about 65% by weight, even more preferably from about 30% by weight to about 60% by weight, relative to the total weight of the composition

The waxes useful for the present invention may be of mineral, fossil, animal, or vegetable origin, hydrogenated oils, or mixtures thereof. Non-limiting examples of waxes include hydrocarbon-based waxes such as polyethylene wax, beeswax, ouricury wax, paraffin, microcrystalline wax, synthetic wax, japan wax, cork fiber waxes or sugar cane waxes, euphorbia cerifera (candelilla) wax, copernicia cerifera (carnauba) wax, lignite waxes, lanolin wax, montan wax, ethylenediamine/stearyl dimer dilinoleate copolymer, hydrogenated oils and glycerides that are solid at 25° C. It is also possible to use silicone waxes, among which mention may be made of alkyl, alkoxy and/or esters of polymethylsiloxane.

Oils which can be used in the invention, mention may be made to polar or slightly polar oils, i.e. oils including an alkyl chain, preferably a C₃-C₄₀ alkyl chain. Non-limiting examples of oils to be used in the present invention include:
linear or branched hydrocarbons such as liquid paraffin, isohexadecane, liquid petroleum jelly and light naphthalene oils, and lanolin, hydrocarbon-based oils of plant origin, such as glyceride triesters, which are generally triesters of fatty acids and of glycerol, the fatty acids of which can have varied chain lengths from C₄ to C₂₄, it being possible for these chains to be saturated or unsaturated and linear or branched; these oils are in particular wheat germ oil, sunflower oil, grape seed oil, sesame oil, corn oil, apricot oil, castor oil, shea oil, avocado oil, olive oil, soybean oil, sweet almond oil, palm oil, rapeseed oil, cottonseed oil, hazelnut oil, macadamia oil, jojoba oil, alfalfa oil, poppy oil, pumpkin seed oil, marrow oil, blackcurrant oil, evening primrose oil, millet oil, barley oil, quinoa oil, rye oil, safflower oil, candlenut oil, passionflower oil and musk rose oil; or also caprylic/capric acid triglycerides,
synthetic esters, for instance oils of formula RCOOR′ in which R represents a linear or branched fatty acid residue containing from 1 to 40 carbon atoms and R′ represents a hydrocarbon-based chain that is especially branched, containing from 1 to 40 carbon atoms, on condition that R+R′ is ≥10, for instance, cetearyl octanoate, isopropyl palmitate, C₁₂-C₁₅ alkyl benzoate, 2-ethylphenyl benzoate, isopropyl lanolate, hexyl laurate, ethylhexyl palmitate, isononyl isononanoate, oleyl erucate, 2-ethylhexyl palmitate, isostearyl isostearate, octanoates, decanoates or ricinoleates of alcohols or polyalcohols, such as propylene glycol dioctanoate; hydroxylated esters, such as isostearyl lactate or diisostearyl malate; and pentaerythritol esters; citrates or tartrates, such as di(linear C₁₂-C₁₃ alkyl) tartrates, and also di(linear C₁₄-C₁₅ alkyl) tartrates, or acetates.
silicone oils such as polydimethylsiloxanes (PDMS's), optionally including a C₃-C₄₀ alkyl or alkoxy chain or a phenyl chain, such as phenyltrimethicones, optionally fluorinated polyalkylmethylsiloxanes, such as polymethyltrifluoropropyldimethylsiloxanes, or with functional groups such as hydroxyl, thiol and/or amine groups; polysiloxanes modified with fatty acids, fatty alcohols or polyoxyalkylenes, fluorosilicones and perfluoro oils;
mixtures thereof.

Non-liming examples of fatty alcohols useful for the present invention are those liquid at room temperature, containing a branched and/or unsaturated carbon-based chain containing from 12 to 26 carbon atoms, for instance octyldodecanol, isostearyl alcohol, behenyl alcohol, oleyl alcohol, 2-hexyldecanol, 2-butyloctanol or 2-undecylpentadecanol.

Non-limiting examples of fatty acids useful for the present invention are higher fatty C₁₂-C₂₂ acids, such as oleic acid, linoleic acid or linolenic acid.

In a preferred embodiment, the additional fatty compounds are selected from the group consisting of synthetic esters, waxes, and mixtures thereof, preferably being selected from the group consisting of ethylhexyl palmitate, diisostearyl malate, polyethylene, and mixtures thereof.

### Additional Ingredients

In addition to the essential components described hereinbefore, the composition of the invention may further comprise any usual cosmetically acceptable ingredient, which may be chosen especially from perfume/fragrance, preserving agents, antioxidants, solvents, actives, vitamins, silicones, polymers, and mixtures thereof.

A person skilled in the art will take care to select the optional additional ingredients and/or the amount thereof such that the advantageous properties of the composition according to the invention are not, or are not substantially, adversely affected by the envisaged addition.

Suitable polymers include, but are not limited to, polyethylene, aluminum starch octenylsuccinate, xanthan gum, poly C₁₀₋₃₀ alkyl acrylate, acrylates/C₁₀₋₃₀ alkyl acrylate crosspolymer, styrene/acrylates copolymer, and mixtures thereof.

Non-limiting example of preserving agent which can be used in accordance with the invention include phenoxyethanol. An example of antioxidant is pentaerythrityl tetra-di-t-butyl hydroxyhydrocinnamate.

Suitable solvents include, but are not limited poly C₁₂₋₁₅ alkyl benzoates, alcohols, glycols and polyols such as glycerin, caprylyl glycol, pentylene glycol, propylene glycol, butylene glycol, and mixtures thereof.

Suitable additional actives include, but are not limited to, disodium EDTA, triethanolamine, and mixtures thereof.

Non-limiting example of vitamins suitable for the composition of the present invention includes tocopherol.

The additional ingredients may represent from 0.1% to 30%, such as from 0.1% to 25% or such as from 0.1 to 20% by weight of the total weight of the composition of the invention. By way of non-limiting illustration, the invention will now be described with reference to the following examples.

EXAMPLES Example 1 A suitable composition according to the present invention was prepared according to Example 1, as follows:

| Function | Ingredient | % wt. |
|---|---|---|
| Emollient | Isononyl isononanoate | 5.0 |
| System | Diisopropyl adipate | 0.5 |
| | Dicaprylyl ether | 5.0 |
| Filler | Silica | 2.0 |
| System | Mica | 1.5 |
| | Talc | 1.5 |
| | Silica silylate | 1.0 |
| UV | Ethylhexyl salicylate | 10.0 |
| System | Drometrizole trisiloxane | 4.0 |
| | Octocrylene | 7.0 |
| | Ethylhexyl triazone | 7.0 |
| | Bis-ethylhexyloxyphenol methoxyphenyl triazine | 4.0 |
| | Butyl methoxydibenzoylmethane | 8.0 |
| Fatty | Isohexadecane | 6.0 |
| compound | Isopropyl palmitate | 7.0 |
| | Paraffin (and) microcrystalline wax (and) synthetic | 3.6 |
| | wax | |
| | Diisopropyl sebacate | 7.0 |
| Fragrance | Fragrance | 0.7 |
| Polymer | Polyethylene | 12.0 |
| Solvent | C₁₂₋₁₅ alkyl benzoate | 7.0 |
| Vitamin | Tocopherol | 0.2 |

A suitable composition according to the present invention was prepared according to Example 2, as follows:

| Function | Ingredient | % wt. |
|---|---|---|
| Emollient | Isononyl isononanoate | 5.0 |
| System | Diisopropyl adipate | 0.5 |
| | Dicaprylyl ether | 5.0 |
| Filler | Silica | 2.0 |
| System | Mica | 1.5 |
| | Silica silylate | 1.0 |
| | Cellulose | 1.5 |
| UV | Ethylhexyl salicylate | 9.0 |
| System | Drometrizole trisiloxane | 4.0 |
| | Octocrylene | 7.0 |
| | Ethylhexyl triazone | 7.0 |
| | Titanium Dioxide | 2.0 |
| | Diethylamino hydroxybenzoyl hexyl benzoate | 14.0 |
| | Bis-ethylhexyloxyphenol methoxyphenyl triazine | 4.0 |
| Fatty | Isohexadecane | 10.0 |
| compound | Isopropyl palmitate | 4.18 |
| | C₁₂-C₁₅ alkyl benzoate | 4.0 |
| | Paraffin (and) microcrystalline wax (and) synthetic | 2.5 |
| | wax | |
| | Diisopropyl sebacato | 4.5 |
| Fragrance | Fragrance | 0.8 |
| Polymer | Polyethylene | 10.0 |
| Pigment | Yellow 6 Lake | 0.12 |
| Preserving | Pentaerythrityl tetra-di-t-butyl hydroxyhydrocinnamate | 0.2 |
| Agent | | |
| Vitamin | Tocopherol | 0.2 |

### SPF and Water Resistance Evaluation

The composition according to Example 1 was submitted to a SPF and a Water Resistance evaluation. The tests were performed following the ISO/EN 24444 Cosmetics Sun protection test-in vivo determination of the Sun Protection Factor (SPF) (2010). The Water resistance evaluation was performed according to the Colipa Guidelines (December 2005). The results are summarized in table 1 below.

TABLE 1

| | |
|---|---|
| PARAMETER | EXAMPLE 1 |

Without Immersion

| | |
|---|---|
| Average SPF Value | 88.8 |

With Immersion

| | |
|---|---|
| Average SPF Value | 48.7 |
| Water Resistance (% WRR) | 54.6% |

It can be seen from the tests performed that example 1 according to the present invention shown high mean SPF value and good water-resistance property.

### Product Performance Evaluation

The objective of this study was to assess and compare the effect on sebum and shininess control of the anhydrous cosmetic composition according to Example 1 vs bare skin until 2 hours of exposure in Hammam room conditions (37±1° C./60±5% RH).

The volunteers arrived without product in the face and stayed in controlled environment for 20 min (21° C.±1; 45%±5 RH) before the first measurements (T0—baseline values), when sebum and shininess levels were evaluated using the equipment's Sebumeter and LightCam and standardized VISIA pictures were taken.

After that, the anhydrous cosmetic composition of Example 1 was randomly applied on half face by the investigator. The volunteers stayed in controlled environment for 10 min (21° C.±1; 45%±5 RH).

Thereafter, sebum and shininess levels were evaluated (T10 min) and new pictures were taken. After 2 h in Hammam room conditions (T2H) the measurements were repeated: shininess and sebum levels were evaluated, and also VISIA pictures.

The anhydrous cosmetic composition of Example 1 showed significant sebum and shine reduction and control ability (i.e., anti-oiliness properties). Also, the anhydrous cosmetic composition of Example 1 showed statistically significant differences compared to the untreated skin considering sebum and shininess 10 minutes after the application and also after 2 hours of exposure to Hammam room with the side treated with the composition of Example 1 presenting the lowest values.

TABLE 2
Average of sebum Casual Level ± confidence interval (CI95%).

| | | T10 min | T2 H (in Hammam |
|---|---|---|---|
| Product | T0 (baseline) | (immediate) | conditions) |
| Example 1 | 210.09 ± 11.30 | 144.25 ± 8.40 | 155.23 ± 9.10 |
| Bare Skin | 204.84 ± 11.71 | 199.86 ± 11.35 | 215.32 ± 6.77 |

The anhydrous cosmetic composition according to Example 1 significantly reduced the sebum casual level 10 minutes after the application (p=0.000). After 2 hours in Hammam room sebum casual level remained significantly lower compared to the baseline (p=0.000) and did not statistically change in comparison to T10 min (p=0.110).

Sebum casual level for the bare skin remained constant 10 minutes after application (T10 min) (p=0.523). After 2 hours in Hammam room there was a significant increase in comparison to T10 min (p=0.005), and the sebum reestablished its initial condition (p=0.068).

TABLE 3
Delta of sebum Casual Level (ΔTi − T0) ±
confidence interval (CI95%).

| Product | T10 min − T0 | T2 H − T0 |
|---|---|---|
| Example 1 | −65.84 ± 12.50 | −54.86 ± 12.84 |
| Bare Skin | −4.98 ± 4.37 | 10.47 ± 11.19 |
| Example 1 - Bare Skin | −28.26% | −31.58% |

The anhydrous cosmetic composition according to Example 1 showed statistically significant differences compared to the untreated bare skin considering sebum casual level 10 minutes after the application (p=0.000) and also after 2 hours of exposure to Hammam room (p=0.000), presenting significant lower values in both times of assessment.

TABLE 4
Average of Gc levels ± confidence interval (CI95%).

| | | T10 min | T2 H (in Hammam |
|---|---|---|---|
| Product | T0 (baseline) | (immediate) | conditions) |
| Example 1 | 44.89 ± 6.28 | 37.79 ± 4.85 | 42.32 ± 4.23 |
| Bare Skin | 43.18 ± 6.34 | 47.00 ± 7.19 | 54.08 ± 6.19 |

The anhydrous cosmetic composition according to Example 1 significantly reduced the shininess 10 minutes after the application compared to the baseline condition (p=0.010). After 2 hours in Hammam room the increase of shininess was not significant compared to T10 min (p=0.149) and also in comparison to the baseline condition (p=0.595).

Shininess for the untreated skin increased 10 minutes after application (T10 min) (p=0.000). After 2 hours in Hammam room there was a significant increase was observed compared to T10 min (p=0.000) and also compared to the baseline condition (T0) (p=0.002).

TABLE 5
Delta of shininess level (ΔTi − T0) ± confidence interval (CI95%).

| Product | T10 min − T0 | T2 H − T0 |
|---|---|---|
| Example 1 | −7.10 ± 4.01 | −2.58 ± 6.05 |
| Bare Skin | 3.82 ± 2.15 | 10.89 ± 3.82 |
| Example 1 - Bare Skin | −22.69% | −29.50% |

The anhydrous cosmetic composition according to Example 1 and the untreated skin showed statistically significant differences between them considering shininess level 10 minutes after the application (p=0.000) and also after 2 hours of exposure to Hammam room (p=0.000), with the side treated by The anhydrous cosmetic composition according to Example 1 presenting lower values.

In comparison to the bare skin, the anhydrous cosmetic composition according to Example 1 was able to reduce shininess 22.69% 10 minutes after application and 29.50% after 2 hours in Hammam room conditions.

### Product Performance Evaluation in Comparison with Conventional Products

The anhydrous cosmetic composition of the present invention, according to Example 1, was evaluated in comparison with a conventional product of the prior art having the following ingredients: cera microcristallina, isononyl isononanoate, nylon-12, homosalate, octocrylene, ceresin, ethylhexyl salicylate, butyl methoxydibenzoylmethane, diethylamino hydroxybenzoyl hexyl benzoate, dimethicone, C₁₂-C₁₅ alkyl benzoate, bis-ethylhexyloxyphenol methoxyphenol methoxyphenyl triazine, ethylhexyl triazone, zinc oxide, phenoxyethanol, cetearyl dimethicone crosspolymer, tocopheryl acetate, BHT, parfum, triethoxycaprylylsilane, and benzyl salicylate.

After the evaluation of the texture, it was observed that the conventional product is more slippery and easier to distribute, however, it crumbles a little on the edges of the stick and has a more waxy touch at the end of the application on the skin. Also, the conventional product leaves a slight blurry look, and it was easy to observe where the product was applied (undesired effect).

Unlike the conventional product of the prior, the anhydrous cosmetic composition of the present invention has more resistance when sliding the stick through the skin and it is difficult to perceive the product being transferred from the bullet to the skin, as the texture is transparent and dull (matte effect), which makes it difficult to perceive the product being transferred from the stick to the skin (desired effect).

In conclusion, the anhydrous cosmetic composition of the present invention, according to Example 1, is less slippery, less silicone and less waxy than the conventional product of the prior art, as well as the anhydrous cosmetic composition of the present invention leaves a transparent film, whereas the conventional product of the prior art leaves a white film.

Regarding the immediate effect, the product of the present invention is drier and has a matte effect. After the return of 4 hours after the application of the product on the skin, the product of the present resulted in more dry touch (medium amount of attribute), touch with light waxy residual and slightly soft. Concerning the visual effect, the matte effect was preserved.

Unlike the present invention, the conventional product of the prior art left the touch slightly sticky, the fingers lock when sliding on the surface of the skin and has a waxy residue—with a lot of presence of this attribute.

In other words, the anhydrous cosmetic composition of the present invention having enhanced anti-oiliness properties, dry touch to the skin after the application combined with mate effect, and with a very light, thin and invisible touch on the skin.

1. An anhydrous cosmetic composition, comprising:
(a) at least 1% by weight of an emollient system, relative to the total weight of the composition;
(b) at least 0.5% by weight of a filler system, relative to the total weight of the composition; and
(c) an UV filter system
wherein the emollient system comprises at least one emollient selected from the group consisting of diisopropyl adipate, isononyl isononanoate, dicaprylyl ether, caprylic/capric triglyceride, dicaprylyl carbonate, isopropyl lauroyl sarcosinate, dibutyl adipate, isoamyl caprate-caprylate and mixtures thereof, and
wherein the filler system comprises at least one filler selected from the group consisting of silica, silica silylate, talc, mica, cellulose and mixtures thereof.

2. The anhydrous cosmetic composition, according to claim 1, wherein the emollient system in the anhydrous cosmetic composition is from 1% by weight to 35% by weight, relative to the total weight of the composition.

3. The anhydrous cosmetic composition, according to claim 1, wherein the filler system in the anhydrous cosmetic composition is from 0.5% by weight to 20% by weight, relative to the total weight of the composition.

4. The anhydrous cosmetic composition, according to claim 1, wherein the UV filter system in the anhydrous cosmetic composition is from 0.1% by weight to 40% by weight, relative to the total weight of the composition.

5. The anhydrous cosmetic composition, according to claim 1, wherein the UV filter comprises at least one UV filter selected from the group consisting of inorganic UV filters, organic UV filters and mixtures thereof.

6. The anhydrous cosmetic composition, according to claim 5, wherein the UV filter system comprises at least one organic UV filter selected from the group consisting of anthranilic compounds; dibenzoylmethane compounds; cinnamic compounds; salicylic compounds; camphor compounds; benzophenone compounds; β,β-diphenylacrylate compounds; triazine compounds; benzotriazole compounds; benzalmalonate compounds; benzimidazole compounds; imidazoline compounds; bis-benzoazolyl compounds; p-aminobenzoic acid (PABA) compounds; methylenebis(hydroxyphenylbenzotriazole) compounds; benzoxazole compounds; screening polymers and screening silicones; dimers derived from a-alkylstyrene; 4,4-diarylbutadienes compounds; guaiazulene and derivatives thereof; rutin and derivatives thereof; flavonoids; bioflavonoids; oryzanol and derivatives thereof; quinic acid and derivatives thereof; phenols; retinol; cysteine; aromatic amino acids; peptides having an aromatic amino acid residue; and mixtures thereof.

7. The anhydrous cosmetic composition, according to claim 5, wherein the UV filter system comprises at least one inorganic UV filter selected from the group consisting of silicon carbide, coated or uncoated metal oxides, and mixtures thereof.

8. The anhydrous cosmetic composition, according to claim 1, further comprising at least one additional fatty compound in an amount ranging from 20% by weight to 70% by weight, relative to the total weight of composition.

9. The anhydrous cosmetic composition, according to claim 8, wherein the at least one additional fatty compound is selected from the group consisting of oils, waxes, fatty acids, fatty alcohols, and mixtures thereof.

10. An anhydrous cosmetic composition, comprising:
(a) from 1% to 35% by weight of an emollient system, relative to the total weight of the composition;
(b) from 0.5% to 20% by weight of a filler system, relative to the total weight of the composition; and
(c) from 0.1% to 40% by weight of an UV filter system, relative to the total weight of the composition.

11. A process of manufacturing an anhydrous cosmetic composition comprising:
at least 1% by weight of an emollient system, relative to the total weight of the composition;
at least 0.5% by weight of a filler system, relative to the total weight of the composition; and
an UV filter system; wherein the process comprises:
(a) mixing the emollient system with the UV filter system;
(b) optionally, incorporating additional ingredients to the mixture of step (a);
(c) incorporating the filler system to the mixture of step (b); and
(d) homogenizing the mixture of step (c).

12. Use of the anhydrous cosmetic composition, as defined in claim 1, for the manufacture of a cosmetic product to be used as a sunscreen daily product.

13. Use of the anhydrous cosmetic composition, as defined in claim 1, for the manufacture of a cosmetic product for reducing the oiliness of the skin.
